(19) United States
(12) Patent Application Publication
(10) Pub. No.: US 2007/0272259 A1
(43) Pub. Date: Nov. 29, 2007

(54) Surgical procedure for inserting a device between anatomical structures

(75) Inventors: Randall Allard, Germantown, TN (US); Kent M. Anderson, Memphis, TN (US); Aurelien Bruneau, Memphis, TN (US); Eric C. Lange, Collierville, TN (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 11/438,763

(22) Filed: May 23, 2006

(51) Int. Cl. A61B 19/00

(52) U.S. Cl. 128/898

(57) ABSTRACT

A surgical procedure according to which a member containing a material is inserted between two anatomical structures; and the member is caused to change state and expand into engagement with at least one of the structures.

### BACKGROUND

The present invention relates to a surgical procedure for inserting a device between anatomical structures and, more particularly, to such a procedure involving expansion of the device after it is inserted.

It is often desirable to insert a device between anatomical structures for several reasons. For example, it can be inserted in a manner so that it engages the structures and serves as an implant for stabilizing the structures and absorbing shock. Alternately, a device can be temporarily inserted between the structures and function to distract the structures to permit another device, such as a prosthesis, to be implanted between the structures. According to another example, a device can be inserted between the structures to distract the structures to permit another surgical procedure to be performed in the space formed by the distraction, after which the device is released and removed.

Although devices have been designed for one or more of the above uses, they are not without problems. For example, it is often difficult to insert the device without requiring excessive invasion of the anatomy, damage to the adjacent anatomical structures, removal of the soft tissue and/or bone, or over-distraction. Embodiments of the present invention improve upon these techniques and various embodiments of the invention may possess one or more of the above features and advantages, or provide one or more solutions to the above problems existing in the prior art.

### BRIEF DESCRIPTION OF THE DRAWINGS

FIG. 1 is a side elevational view of an adult human vertebral column.

FIG. 2 is a posterior elevational view of the column of FIG. 1.

FIG. 3 is an elevational view of one of the vertebrae of the column of FIGS. 1 and 2.

FIG. 4A-4C are elevational views depicting a device for insertion in the column of FIGS. 1-3.

FIG. 5A-5C are enlarged, partial, isometric views of a portion of the column of FIGS. 1 and 2, including the lower three vertebrae of the column, and depicting a procedure for inserting the device of FIGS. 4A-4D between two adjacent vertebrae.

FIG. 6 is a view similar to that of FIG. 4C, but depicting an alternate embodiment of the device.

### DETAILED DESCRIPTION

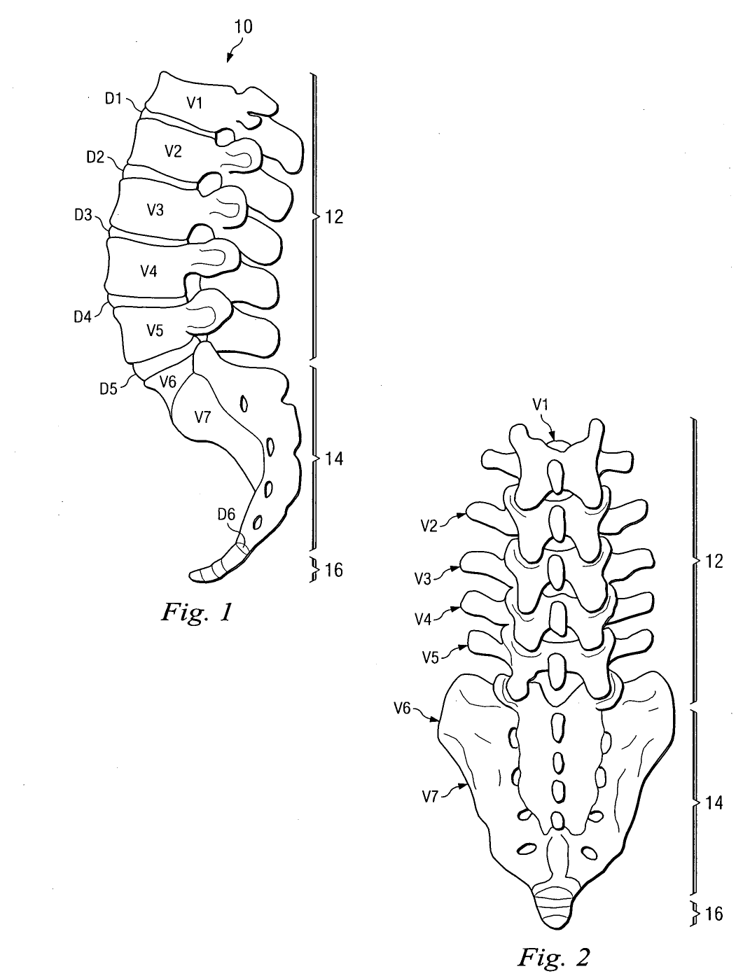

With reference to FIGS. 1 and 2, the reference numeral 10 refers, in general, to the lower portion of a human vertebral column. The column 10 includes a lumbar region 12, a sacrum 14, and a coccyx 16. The flexible, soft portion of the column 10, which includes the thoracic region and the cervical region, is not shown.

The lumbar region 12 includes five vertebrae V1, V2, V3, V4 and V5 separated by intervertebral discs D1, D2, D3, and D4, with the disc D1 extending between the vertebrae V1 and V2, the disc D2 extending between the vertebrae V2 and V3, the disc D3 extending between the vertebrae V3 and V4, and the disc D4 extending between the vertebrae V4 and V5.

The sacrum 14 includes five fused vertebrae, one of which is a superior vertebrae V6 separated from the vertebrae V5 by a disc D5. The other four fused vertebrae of the sacrum 14 are referred to collectively as V7. A disc D6 separates the sacrum 14 from the coccyx 16, which includes four fused vertebrae (not referenced).

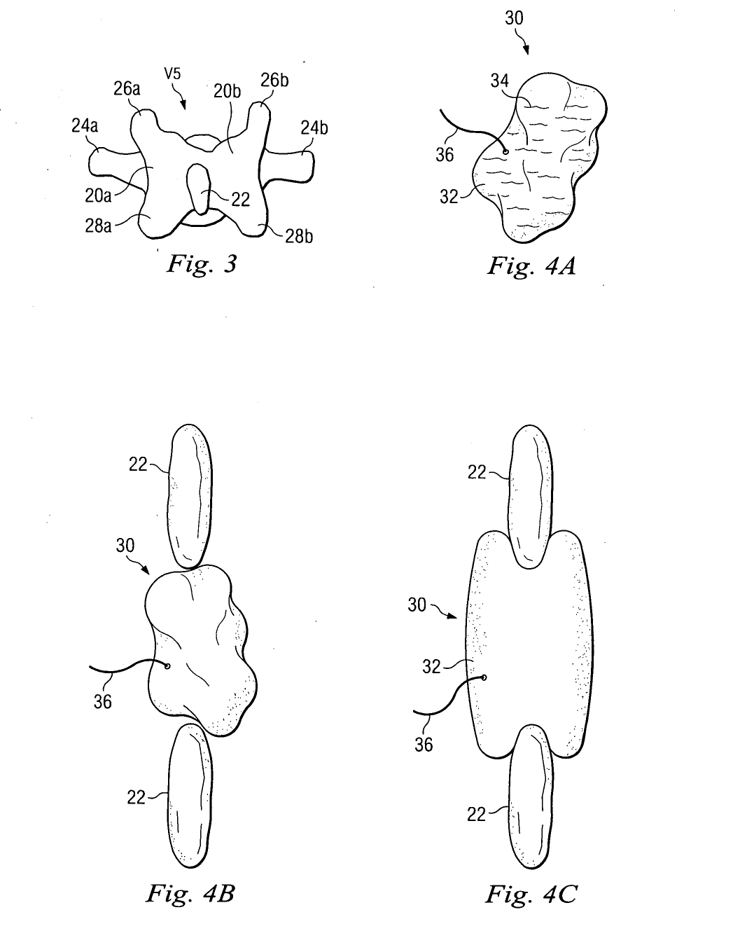

With reference to FIG. 3, the vertebrae V5 includes two laminae 20a and 20b extending to either side (as viewed in FIG. 2) of a spinous process 22 that extends posteriorly from the juncture of the two laminae. Two transverse processes 24a and 24b extend laterally from the laminae 20a and 20b, respectively; two articular processes 26a and 26b extend superiorly from the laminae 20a and 20b respectively; and two articular processes 28a and 28b extend inferiorly from the laminae 20a and 20b, respectively. The inferior articular processes 28a and 28b rest in the superior articular process of the vertebra V2 to form a facet joint. Since the vertebrae V1-V4 are similar to the vertebrae V5, and since the vertebrae V6 and V7 are not necessarily involved in the present invention, they will not be described in detail.

It will be assumed that, for one or more of the reasons set forth above, the vertebrae V3 and V4 are not being adequately supported by the disc D4, and that it is therefore necessary to provide supplemental support and stabilization of these vertebrae. To this end, and referring to FIGS. 4A-4C, a device 30 according to an embodiment of the invention is provided for implantation between the respective spinous processes 22 of the vertebrae V3 and V4.

Referring specifically to FIG. 4A, the device 30 is in the form of a pliable, hollow shell 32 fabricated from a soft flexible material and filled with a fluid 34. To this end, one end of a tube 36 is connected to the shell 32 and the other end (not shown) is connectable to a source of the fluid to permit a selected volume of the fluid to be introduced into the shell 32.

FIG. 4B depicts the device 30 of FIG. 4A inserted between two processes 22 and before it undergoes any expansion in accordance with the following techniques.

The fluid 34 that is introduced into the shell 32 can be one of several types, examples of which are as follows:

1. A fluid that changes to a solid material due to one of the following reactions:
a. A chemical reaction involving one or more of the following fluids—polyurethane, silicone, polyacrylate, epoxy, polymide and phenolic. An example would be a two-part polymer such as silicone or a two part epoxy with one part contained in the shell 32 and the other part being injected into the shell through the tube 36 after the device is inserted between the processes 22. The injection of the other part causes the materials to mix and change to a solid.
b. A chemical reaction involving, for example, a fluid that is catalyzed by an external energy source, examples of which include gamma irradiation, ultra-violet light emitting diodes placed next to the shell 32 or injected in the shell, radio-isotopes, radiation used in cancer therapy, induction heating, polyester resins, water, oxygen, nitrogen argon, helium, hydrogen, peroxides, enzymatic attack (cross linking). Examples of fluids that can be catalyzed in the above manners include polyurethane, silicone, polyacrylate and epoxy.
c. A spray/injected catalyst/activator/accelerator such as the type used for two part cyanoacrylates (marketed under the brand “superglue”). The catalyzation causes the fluid to change to a solid.
d. A thermal-mechanical reaction involving, for example, a polymer or rubber that is introduced, in liquid form, into the shell 32 at an elevated temperature and allowed to cool to ambient temperature, which causes the polymer or rubber to change to a solid.

2. A fluid that changes to a solid material due to one of the following external stimuli in the form of a focused energy source:
a. Utrasonic vibration.
b. Electromagnetic waves.
c. External heat source.
d. Irradiation.
e. Ultrasound
f Radio frequency energy.

In each case, the fluid would consist of an agent, or a solution of agents, such as two-part curing polymers, in the form of silicones, epoxies or the like, that are injected into the shell and subjected to one of the above stimuli to react endothermically and change to a solid.

3. A fluid that is in the form of a material that can be cured by a curing method. Examples of the curing materials are epoxy, acrylate, polyurethane, poluyurea, room temperature vulcanizer, polyvinyl alcohol, and moisture curing silicone. Examples of curing methods are perozides, moisture initiated multipart mix and deliver, focused energy.

FIG. 4C depicts the device 30 after the fluid 34 has changed state and therefore expanded in accordance with any of the above examples. In this case the axial expansion is greater than the distance between the two processes 22, thus causing the respective ends of the device to wrap around corresponding portions of the respective processes.

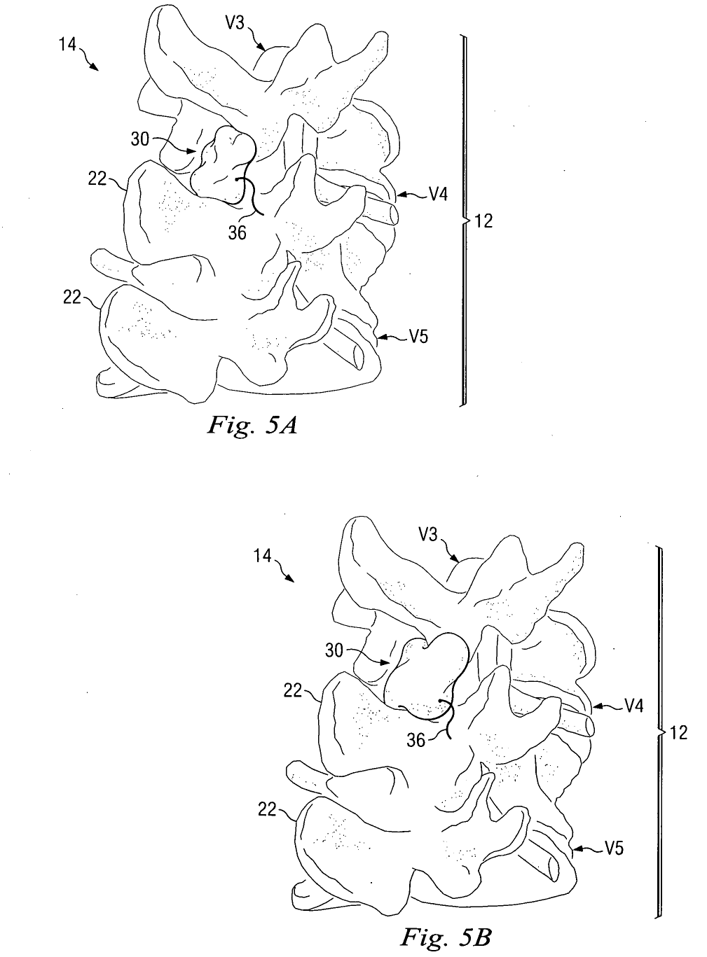

Referring to FIG. 5A, the device 30, in its unexpanded state, is inserted between the respective spinous processes 22 of the vertebrae V3 and V4. Then the fluid 34 is caused to change state to a solid in accordance with any of the foregoing manners which causes the device 30 to take an intermediate state of expansion shown in FIG. 5B, and then a final solid state shown in FIG. 5C. In the last position, the device 30 engages the spinous processes 22 of the vertebrae V3 and V4, respectively, with enough force to firmly secure the device between the processes and stabilize the vertebrae. It is understood that, in moving from the position of FIGS. 5B to 5C, the device 30 can distract, or engage and move, at least one of the processes 22 if it is desired to establish a predetermined spatial relationship between the processes.

In addition to stabilizing the vertebrae V3 and V4, the relatively flexible, soft material of the device 30 readily conforms to the processes and provides excellent shock absorption and deformability, resulting in an improved fit.

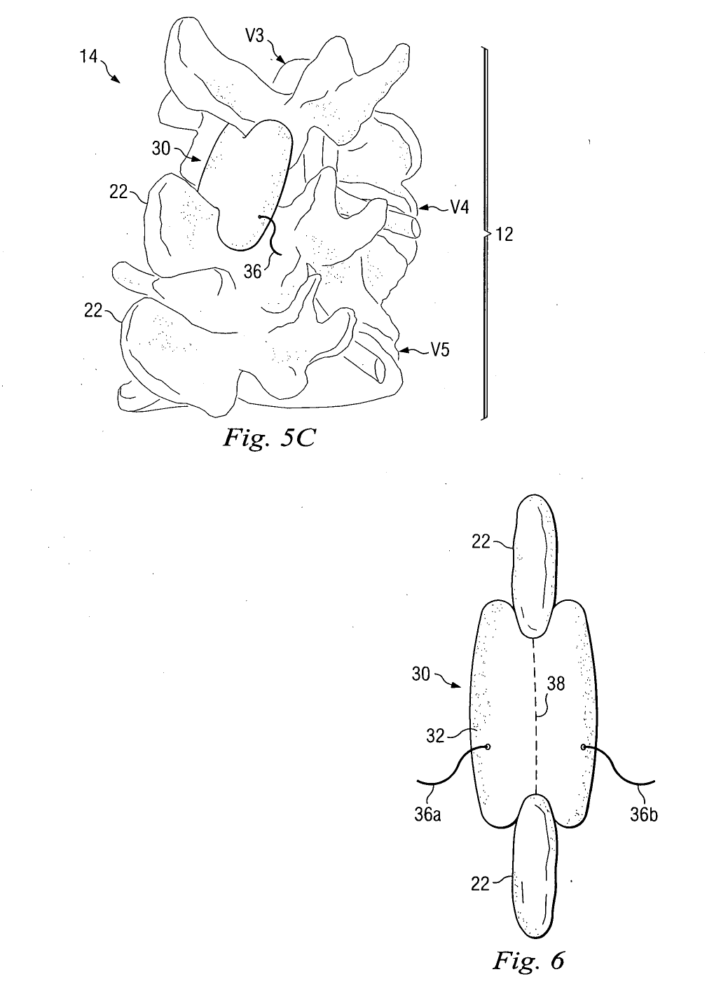

According to an alternate embodiment shown in FIG. 6, a membrane 38 extends through the interior of the shell 32′ to divide it into two substantially equal chambers 38a and 38b. Two tubes 36a and 36b are connected to the chambers 38a and 38b, respectively, and the tubes are also respectively connected to two sources of fluid to permit a selected volume of the fluids to be introduced into the chambers 38a and 38b. For example, a two-part curing polymer, such as silicone or epoxy in liquid form, could be used with the two parts being introduced into the chambers 38a and 38b, respectively and maintained separately by the membrane 38.

The membrane 38 is adapted to break in response to the application of an electrical signal or an external mechanical force, in a conventional manner, under conditions to be described.

In operation, the shell 32′ is inserted between the processes 22 in the same manner as discussed above and shown in FIGS. 5A-5V. The electrical signal or external mechanical force discussed above is then is applied to the membrane 38 to cause it to break so that the two fluids can mix and form a solid. As in the previous embodiments, it is understood that the design is such that, after the fluid 34 changes to a solid in accordance with the above, the solid material would be of a strength that is sufficient to carry the compressive loads that are placed on it after the shell 32′ is inserted between two processes 22. It is understood that the design is such that, after the fluid 34 changes to a solid in accordance with any of the above embodiments, the change in volume would cause it to fill the shell 32 in a manner so that the device 30 would take a prescribed shape.

According to another alternate embodiment, a solid material is placed in the shell 32 that is of the type that changes state to a gas in response to an external stimulant, or the introduction of a chemical, or the like, into the interior of the shell. This change of state will cause an increase in volume of the solid as it changes to a gas and an attendant expansion of the shell 32.

As in the previous embodiments, it is understood that the design is such that, after the solid changes to a gas in accordance with the above, the change in volume causes it to fill, and/or stretch the shell 32 in a manner so that it takes a prescribed shape. Also, the device 30 thus formed would be of a strength that is sufficient to carry the compressive loads that are placed on it after it is inserted between the two processes 22.

It is understood that the term “expand,” as used throughout this specification, is meant to cover the situation in which the shell 32 is expanded and/or inflated in accordance with all of the above examples and embodiments.

It is also understood that in each of the above embodiments, when the shell 32 is filled with a fluid, such as a gas or liquid, that changes state into a solid in accordance with the foregoing, it will not necessarily expand the shell and cause distraction of the processes 22. In this case, if needed, the surgeon can manually distract the processes 22 prior to insertion of the shell 32 so that, when the fluid changes state to a hard solid it will carry the compressive loads that will be placed on it. Alternately, expansion or distraction may not be needed in some situations, such as when the shell is inserted in the above manner when the patient is in a prone position and the processes apply a compressive load to the shell when the patient is in an upright position.

It is also understood that, in each of the above embodiments, the device 30 does not necessarily have to function as an implant between two processes 22 as described in the examples above, but rather can be used in other different procedures and in other different areas of the anatomy. For example, the device 30 can be inserted between two anatomical structures, such as the processes used in the above examples, and expanded to an extent that it engages and distracts, or moves, one or both of the structures in a direction away from each other, to permit another device, such as a prosthesis, to be implanted between the structures or in an area near the structures. According to another example, the device 30 can be inserted between the structures and expanded to an extent that it engages and distracts the structures to permit another surgical procedure to be performed in the space formed by the distraction. In each of these examples, the device would be released and removed after the procedure is completed.

### Variations

It is understood that variations may be made in the foregoing without departing from the invention and examples of some variations are as follows:
(1) The device 30 and the shell 32 can take shapes that are different from the examples disclosed above.
(2) Rather than inject a second fluid into the shell 32′ to react with a first fluid, the second fluid could be placed in a time release capsule that is inserted into the shell with the first fluid. After a predetermined length of time it would release from the capsule and mix with the first fluid in the manners described above.
(3) The device 30 can be inserted in other areas of the anatomy such as, for example, in an intervertebral disc space.
(4) The device 30 can be inserted between two vertebrae following a corpectomy in which at least one vertebrae is removed.
(5) The types of fluid introduced into the shell 32 can vary.
(6) The types of chemicals and stimuli used to cause the fluid or solid in the shell 32 to change state can vary.
(7) The expansion of the device 30 can be such that it engages only one of the anatomical structures.
(8) Any spatial references made above, such as “under”, “over”, “between”, “upper”, “lower”, “top”, “bottom”, etc., are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw are equivalent structures.

What is claimed is:

1. A surgical procedure comprising:
inserting a member containing a material between two anatomical structures; and
causing the material to change state.

2. The procedure of claim 1 wherein the change of state causes the material to expand into engagement with at least one of the structures.

3. The procedure of claim 2 wherein the expansion causes distraction of the structures.

4. The procedure of claim 1 wherein the material changes from a fluid to a solid material.

5. The procedure of claim 4 further comprising the step of distracting the structures prior to the step of inserting.

6. The procedure of claim 1 wherein the material changes state due to a chemical reaction.

7. The procedure of claim 6 further comprising introducing a reagent into the member that reacts with the fluid to cause the chemical reaction.

8. The procedure of claim 1 wherein the material is a fluid and the fluid changes state to a solid.

9. The procedure of claim 8 wherein the step of causing comprises applying a mechanical force to the fluid to cause it to change state.

10. The procedure of claim 8 wherein the step of causing comprises applying heat to the fluid to cause it to change state.

11. The procedure of claim 1 wherein the material changes state due to a thermal-mechanical chemical reaction.

12. The procedure of claim 1 wherein the material changes state due to an external stimuli.

13. The procedure of claim 12 wherein the external stimuli is an electrical signal.

14. The procedure of claim 12 wherein the external stimuli is an ultrasonic signal.

15. The procedure of claim 12 wherein the external stimuli is a mechanical means.

16. The procedure of claim 12 wherein the external stimuli is electromagnetic waves.

17. The procedure of claim 12 wherein the external stimuli is a thermal source.

18. A surgical procedure comprising:
inserting a member containing a solid material between two anatomical structures; and
causing the solid material to change to a gas and expand into engagement with at least one of the structures.

19. The procedure of claim 18 wherein the solid material changes to a gas due to an external stimuli.

20. The procedure of claim 18 wherein the solid material changes to a gas due to a chemical reaction.

21. The procedure of claim 20 further comprising introducing a chemical to the member to cause the chemical reaction.
